(19) United States
(12) Patent Application Publication
(10) Pub. No.: US 2018/0207017 A1
(43) Pub. Date: Jul. 26, 2018

(54) REHABILITATION AID

(71) Applicant: SLINGTEE, LLC, Spring, TX (US)

(72) Inventors: Michael Samir George, Spring, TX (US); Ami Marie Hazel, Spring, TX (US)

(21) Appl. No.: 15/879,020

(22) Filed: Jan. 24, 2018

Related U.S. Application Data
(60) Provisional application No. 62/450,335, filed on Jan. 25, 2017.

(51) Int. Cl. A61F 5/37; A41B 1/08

(52) U.S. Cl. CPC A61F 5/373; A61F 5/3738; A41B 1/08

(57) ABSTRACT

A rehabilitation aid for use by persons having a need for a sling. The rehabilitation aid can be incorporated into an article of clothing and have a sleeve positioned beneath an arm of a user. The sleeve can be configured to support at least a portion of the weight of the arm and help to substantially immobilize, constrain, or restrict the movement of the upper arm. An adjustable attachment can be included to adjust arm angle. Reinforcements can also be incorporated to help distribute supported weight throughout the article of clothing.

### CROSS REFERENCE TO RELATED APPLICATIONS

The present application claims priority to and the benefit of co-pending U.S. Provisional Patent Application Ser. No. 62/450,335 filed on Jan. 25, 2017, titled “A SHIRT WITH A POCKET THAT FUNCTIONS AS AN ARM SLING”. This reference is incorporated herein in its entirety.

### FIELD

The present disclosure generally relates to a rehabilitation aid for persons having a need for a sling or to immobilize a portion of the arm.

### BACKGROUND

Various injuries and surgical procedures require a person to make use of a sling afterward in order to relieve pressure and weight on the shoulder, immobilize a portion of the arm, or to position the arm properly for recovery.

It is often necessary, especially after a surgical procedure on the shoulder, to restrict the movement of a person's arm to speed the recovery process, as well as relieve the weight and pressure on a person's shoulder.

Recovering from a shoulder surgery often requires that the upper arm of a patient not be moved substantially away from the torso in order to avoid strain on the shoulder. Further, in instances where a cast is worn, this places considerable strain on the shoulder due to the additional weight of the cast.

A sling is often utilized for these purposes, as well as in instances in which a cast is placed on a person's arm. While a sling serves the purpose of relieving weight from the shoulder and substantially immobilizing the arm, presently utilized arm slings are often uncomfortable, do not efficiently perform the function of immobilizing or restricting the motion of at least a portion of the arm, and are aesthetically displeasing.

Slings will often pull on the neck or shoulder of a user causing discomfort or pain when worn.

The present disclosure cures the above deficiencies.

### BRIEF DESCRIPTION OF THE DRAWINGS

The detailed description will be better understood in conjunction with the accompanying drawings as follows:

FIGS. 1A and 1B depict an embodiment of the rehabilitation aid.

FIGS. 2A and 2B depict an embodiment of the rehabilitation aid.

FIGS. 3A and 3B depict an embodiment of the rehabilitation aid.

The embodiments of the present disclosure are detailed below with reference to the listed Figures.

### DETAILED DESCRIPTION OF THE EMBODIMENTS

Before explaining the present disclosure in detail, it is to be understood that the disclosure is not limited to the specifics of particular embodiments as described and that it can be practiced, constructed, or carried out in various ways.

While embodiments of the disclosure have been shown and described, modifications thereof can be made by one skilled in the art without departing from the spirit and teachings of the disclosure. The embodiments described herein are exemplary only and are not intended to be limiting.

Specific structural and functional details disclosed herein are not to be interpreted as limiting, but merely as a basis of the claims and as a representative basis for teaching persons having ordinary skill in the art to variously employ the present embodiments. Many variations and modifications of embodiments disclosed herein are possible and are within the scope of the present disclosure.

Where numerical ranges or limitations are expressly stated, such express ranges or limitations should be understood to include iterative ranges or limitations of like magnitude falling within the expressly stated ranges or limitations.

The use of the word “a” or “an” when used in conjunction with the term “comprising” in the claims and/or the specification may mean “one,” but it is also consistent with the meaning of “one or more,” “at least one,” and “one or more than one.”

The use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, and the like.

Accordingly, the scope of protection is not limited by the description herein, but is only limited by the claims which follow, encompassing all equivalents of the subject matter of the claims. Each and every claim is hereby incorporated into the specification as an embodiment of the present disclosure. Thus, the claims are a further description and are an addition to the embodiments of the present disclosure.

The inclusion or discussion of a reference is not an admission that it is prior art to the present disclosure, especially any reference that may have a publication date after the priority date of this application. The disclosures of all patents, patent applications, and publications cited herein are hereby incorporated by reference, to the extent they provide background knowledge; or exemplary, procedural or other details supplementary to those set forth herein.

The embodiments of the present disclosure generally relate to a rehabilitation aid for persons having a need for a sling and/or to immobilize at least a portion of the arm, such as due to shoulder or arm injuries and/or recovering from a surgery.

The present disclosure cures the deficiencies of prior art slings by aiding to immobilize the upper arm in a certain fashion, while being incorporated into an article of clothing in order to be aesthetically pleasing. Further, the present embodiments eliminate a pulling on the neck or shoulder of the user, thereby leading to a much more comfortable experience for the user

The present disclosure relates to a rehabilitation aid comprising an article of clothing with an opening for a left arm, and an opening for a right arm of the user. A sleeve is attached to the article of clothing below either the opening for the left arm, or the opening for the right arm of the user.

The sleeve is configured to support at least a portion of the weight of an arm of the user and to restrict motion of an upper arm of the user such that the upper arm cannot substantially be moved away from the torso of the user. In embodiments, a stretchable attachment or sleeve material can be utilized to give the sleeve a limited range of motion.

The rehabilitation aid can be used in any instance in which a user has the need for a sling, such as after shoulder surgeries, and/or when the user has a cast on the arm. The rehabilitation aid can relieve pressure on the shoulder, as well as constrain the movement of the upper arm in order to speed recovery. Various embodiments can allow for adjustment of the sleeve angle for comfort and/or as directed by a physician to aid in recovery.

The article of clothing can be any article worn by a person on the upper body, such as a t-shirt, a button-down or dress shirt, a blouse, a vest, a jacket, and the like. The rehabilitation aid can be configured to hide much of the support structure in order to make the article of clothing aesthetically pleasing as well as comfortable.

In embodiments, the article of clothing can be made from fabric which does not stretch substantially. In other embodiments, articles of clothing made from fabric which stretches can be reinforced to support the rehabilitation aid.

A sleeve can be attached to the article of clothing below an opening for either the left arm or the right arm of a user. Various means of attaching the sleeve can be utilized by persons having ordinary skill in the art, based upon specific needs of the user, or for aesthetic purposes.

Exemplary attachment means can include, but are not limited to: a stitch, a button, a zipper, a toggle fastener, a ball and socket snap closure, a buckle, a braided loop fastener, a frog fastener, a hook and clip fastener, a clasp fastener, a grommet fastener, a hook and eye closure, an adhesive, or a hook and loop fastener. In embodiments, multiple attachment means can be utilized for fine positioning adjustments, such as a toggle fastener in conjunction with a hook and loop fastener.

The sleeve can be configured to support at least the elbow and keep the elbow close to the torso of the user by being attached to the article of clothing. This serves to also constrain the movement of the upper arm, thereby aiding in recovery after shoulder surgery and/or shoulder injuries. The sleeve can also be configured to support at least some of the weight of the arm, thereby reducing the strain applied to the shoulder. This can especially be helpful when a cast is being worn by the user. The article of clothing or any utilized reinforcements can help to distribute the supported weight of the arm over a large area, thereby also reducing pulling or pinching in the neck area, as is prevalent with presently used slings.

In embodiments, the sleeve is positionable on the article of clothing. This can be accomplished by using multiple attachment means, or a single means with various attachment positions. For example, the sleeve can be attached using a hook and loop fastener with a large area of attachment.

In embodiments, the sleeve can further comprise an adjustable attachment for at least a portion of the sleeve. This adjustable attachment can support the sleeve away from the elbow, i.e. closer to the wrist of the user than the attachment of the sleeve to the article of clothing. The adjustable attachment can be used to adjust the arm angle of the user for medical or comfort purposes. The adjustable attachment may have various usable points of attachment, or itself be adjustable in length. Various buckles, rings, hooks, clips, slides, and the like can be incorporated in order to make length adjustments to the adjustable attachment.

In embodiments, the adjustable attachment is detachable from the article of clothing to allow for free movement of the user's forearm, while still constraining the movement of the user's elbow and upper arm.

In embodiments, the rehabilitation aid can comprise a first reinforcement extending substantially superior to the sleeve, i.e. toward the head and/or shoulders of the user. This can aid in supporting the weight of the user's arm and/or cast. In embodiments, the first reinforcement to or over the shoulder of the user.

The rehabilitation aid can comprise a second reinforcement extending substantially laterally to the sleeve, such as at least partially around the torso of a user. This can aid in keeping the elbow, and therefore the upper arm, close to the torso of the user. In embodiments, the second reinforcement can extend partially or completely around the torso of the user.

In embodiments, both the first reinforcement and the second reinforcement can be utilized. In embodiments, one reinforcement or both reinforcements can be internal to the article of clothing, such that they are not externally visible when viewing the article of clothing.

A kit can be provided for converting an article of clothing into a rehabilitation aid. The kit can comprise a sleeve, an adjustable attachment, optionally a first reinforcement, and optionally a second reinforcement.

Detailed instructions on proper positioning of the sleeve for attachment to the article of clothing can be provided in conjunction with the kit.

Various means of attachment as discussed above can be provided for attachment to an article of clothing. In embodiments, a fabric adhesive can be utilized to allow for the kit components to be removed when no longer necessary.

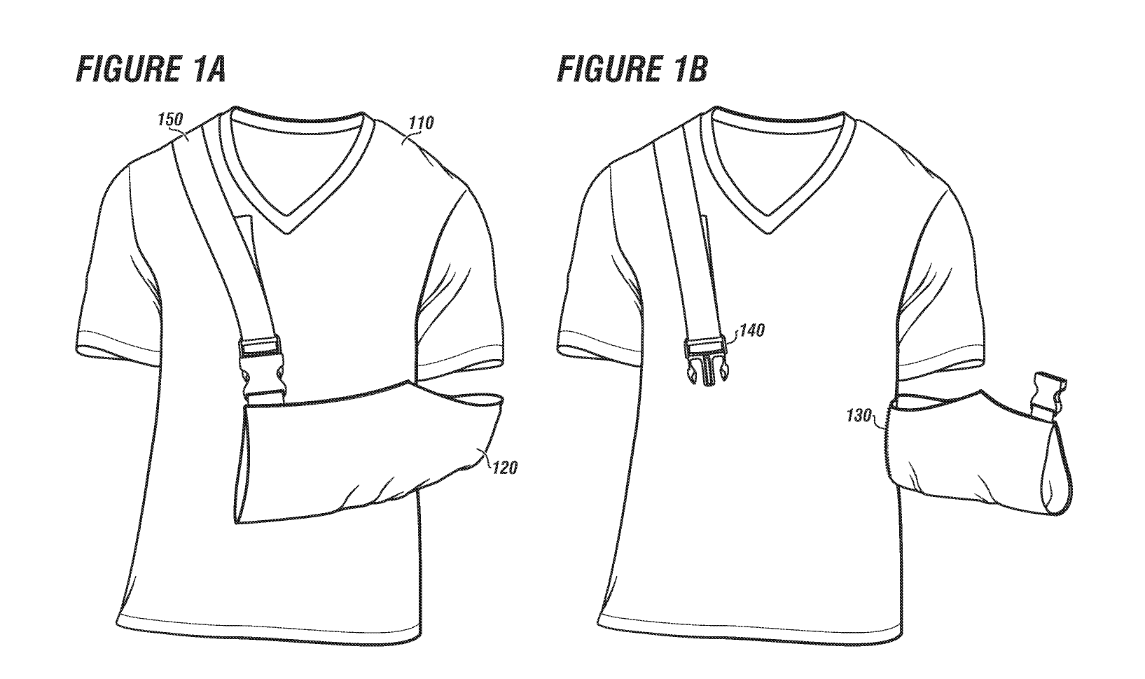

Turning now to the Figures, FIGS. 1A and 1B depict an embodiment of the rehabilitation aid.

In this embodiment, article of clothing 110, shown here as a t-shirt has a sleeve 120 attached below an opening for the left arm of the user. The sleeve 120 can be positioned such that it supports at least a portion of the weight of the user's arm.

In this embodiment, the attachment 130 of the sleeve 120 is by being sewn to the article of clothing 110. Various other attachments, as discussed above can be utilized. The attachment 130 also constrains and restricts the motion of the user's upper arm, holding the upper arm close to the body. The sleeve 120 can be shaped so as to receive an elbow of the user at one end near the attachment 130.

The sleeve 120 can have an adjustable attachment 140. The adjustable attachment 140 can be used to adjust the arm angle of the user for user comfort, or as directed by a physician. In the embodiment shown, the adjustable attachment 140 is detachable to allow for the user to have free range of motion of the forearm, while still maintaining the upper arm close to the torso, as would be desirable after a shoulder injury or shoulder surgery. In this embodiment, a first reinforcement 150 can be incorporated with the adjustable attachment 140 and secured proximate the shoulder opposite the arm beneath which the sleeve 120 is attached.

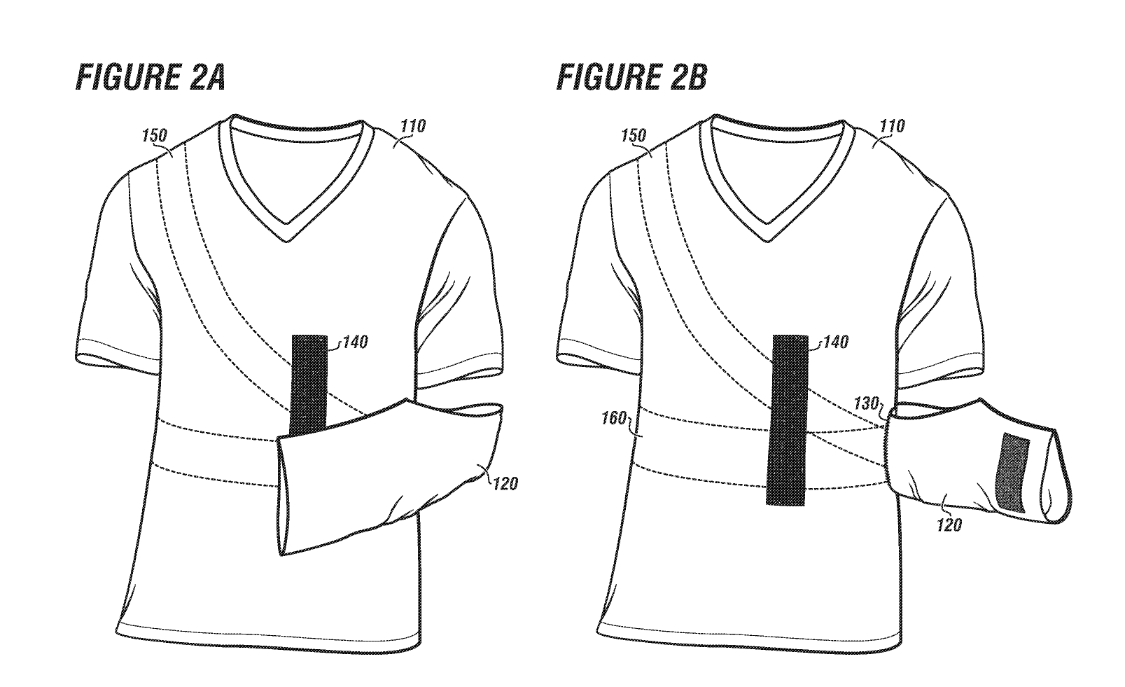

FIGS. 2A and 2B depict an embodiment of the rehabilitation aid.

In this embodiment, the first reinforcement 150 extends substantially superior to the sleeve 120. In the embodiment shown, the first reinforcement 150 can be secured at the attachment 130 to provide support for the sleeve 120. This can help to aid in supporting the weight of the arm, as well as distribute the weight such that the article of clothing 110 does not pull on the user at the attachment 130.

In embodiments, the second reinforcement 160 can also be secured at the attachment 130 to provide support for the sleeve 120. This can help to ensure that the article of clothing 110 does not pull on the user at the attachment 130. In embodiments, the reinforcements can be secured to the article of clothing such that they are not externally visible.

The adjustable attachment 140 for the sleeve is shown here as a hook and loop fastener with a large range of attachment points for adjusting an arm angle of the user.

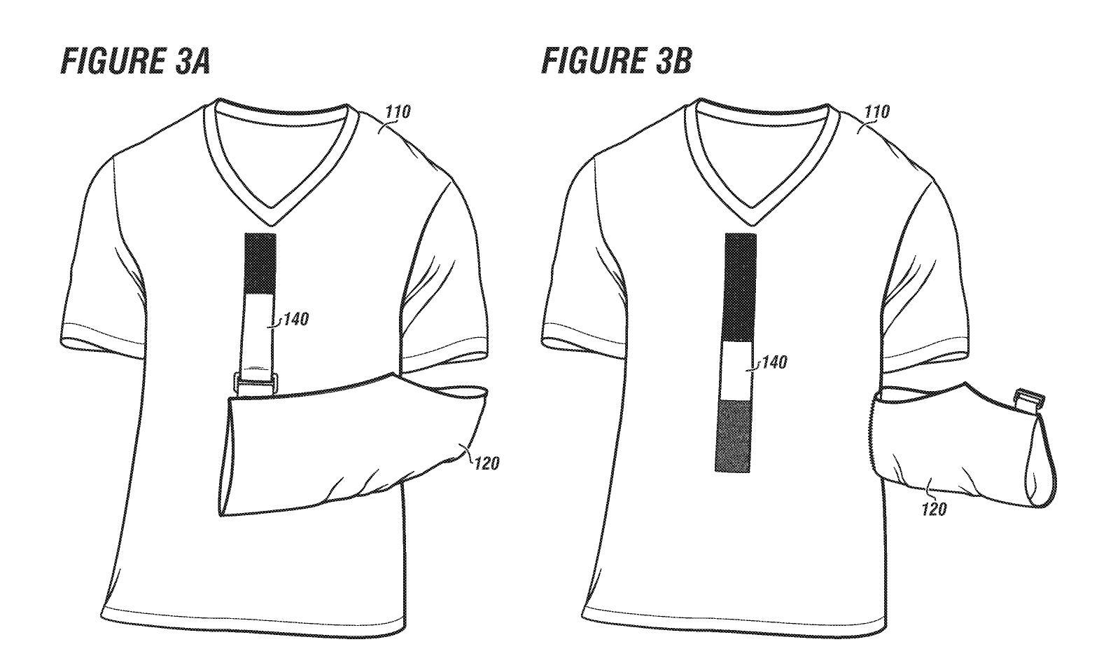

FIGS. 3A and 3B depict an embodiment of the rehabilitation aid.

Here the adjustable attachment 140 is secured to the front of the article of clothing 110.

While the present disclosure emphasizes the embodiments, it should be understood that within the scope of the appended claims, the disclosure might be embodied other than as specifically described herein.

What is claimed is:

1. A rehabilitation aid comprising:
a. an article of clothing covering at least a portion of a torso of a user comprising an opening for a left arm, and an opening for a right arm of the user; and
b. a sleeve attached to the article of clothing below either the opening for the left arm, or the opening for the right arm of the user; and
wherein the sleeve is configured to support at least a portion of the weight of an arm of the user and to restrict motion of an upper arm of the user such that the upper arm cannot substantially be moved away from the torso of the user.

2. The rehabilitation aid of claim 1, further comprising an adjustable attachment for at least a portion of the sleeve.

3. The rehabilitation aid of claim 1, further comprising a first reinforcement extending substantially superior to the sleeve.

4. The rehabilitation aid of claim 1, further comprising a second reinforcement extending substantially laterally to the sleeve.

5. The rehabilitation aid of claim 1, further comprising a first reinforcement extending substantially superior to the sleeve and a second reinforcement extending substantially laterally to the sleeve.

6. The rehabilitation aid of claim 1, wherein the sleeve is attached utilizing:
a. a stitch;
b. a button;
c. a zipper;
d. a toggle fastener;
e. a ball and socket snap closure;
f. a buckle;
g. a braided loop fastener;
h. a frog fastener;
i. a hook and clip fastener;
j. a clasp fastener;
k. a grommet fastener;
l. a hook and eye closure;
m. a magnet;
n. an adhesive;
o. a hook and loop fastener; or
P. combinations thereof.

7. The rehabilitation aid of claim 1, wherein the sleeve is positionable on the article of clothing.

8. The rehabilitation aid of claim 1, wherein the sleeve is shaped to receive an elbow of the user at an end of the sleeve.

9. The rehabilitation aid of claim 2, wherein the adjustable attachment is detachable.

10. The rehabilitation aid of claim 3, wherein the first reinforcement extends to at least the shoulder of the user.

11. The rehabilitation aid of claim 4, wherein the second reinforcement extends at least partially around the torso of the user.

12. The rehabilitation aid of claim 5, wherein the first reinforcement extending substantially superior to the sleeve and the second reinforcement extending substantially laterally to the sleeve are not externally visible when viewing the article of clothing.

13. A rehabilitation aid comprising:
a. an article of clothing covering at least a portion of a torso of a user comprising an opening for a left arm, and an opening for a right arm of the user;
b. a sleeve attached to the article of clothing below either the opening for the left arm, or the opening for the right arm of the user; and
c. an adjustable attachment detachably secured to the sleeve and secured proximate an opposite shoulder of the user, wherein the adjustable attachment also functions as a first reinforcement; and
wherein the sleeve is configured to support at least a portion of the weight of an arm of the user and to restrict motion of an upper arm of the user such that the upper arm cannot substantially be moved away from the torso of the user, and further wherein the adjustable attachment is configured to support at least a portion of the weight of the arm of the user.

14. A kit for converting an article of clothing to a rehabilitation aid comprising:
a. a sleeve for attaching to the article of clothing;
b. an adjustable attachment for the sleeve;
c. optionally, a first reinforcement; and
d. optionally, a second reinforcement.
